(19) United States
(12) Patent Application Publication
(10) Pub. No.: US 2025/0099059 A1
(43) Pub. Date: Mar. 27, 2025

(54) HEMODYNAMIC ASSESSMENT OF AORTIC VALVE STENOSIS BY CARDIAC COMPUTED TOMOGRAPHY

(71) Applicant: Valley Health System, Paramus, NJ (US)

(72) Inventor: Himanshu Gupta, Ridgewood, NJ (US)

(73) Assignee: Valley Health System, Paramus, NJ (US)

(21) Appl. No.: 18/894,566

(22) Filed: Sep. 24, 2024

Related U.S. Application Data
(60) Provisional application No. 63/585,623, filed on Sep. 27, 2023.

(51) Int. Cl. A61B 6/50; A61B 6/00; A61B 6/03

(52) U.S. Cl. CPC A61B 6/503; A61B 6/035; A61B 6/507; A61B 6/5217; A61B 6/54

(57) ABSTRACT

Systems and methods for performing hemodynamic assessments of aortic valve stenosis by cardiac computed tomography (CT) scanning are provided. The method may comprise performing a cardiac CT scan of a patient and, using a computing device, determining, via the CT scan, an aortic valve area (AVA_{CT}) of the patient and a left ventricular outflow tract area (LVOT Area_{CT}) of the patient, calculating an anatomic dimensionless index (DI_{CT}) based on the aortic valve area (AVA_{CT}) and the left ventricular outflow tract area (LVOT Area_{CT}), and determining, based on the aortic valve area (AVA_{CT}), the left ventricular outflow tract area (LVOT Area_{CT}), and the anatomic dimensionless index (DI_{CT}), a severity level of aortic valve stenosis (AS) of the patient.

### CROSS REFERENCE TO RELATED APPLICATIONS

This application claims priority to U.S. Provisional patent Application No. 63/585,623, filed on Sep. 27, 2023, the content of which is incorporated herein by reference in its entirety.

### TECHNICAL FIELD

Embodiments of the present disclosure relate to systems and methods for performing hemodynamic assessments of aortic valve stenosis by cardiac computed tomography.

### BACKGROUND

Echocardiography is an imaging technique whereby ultrasounds are used to image the heart, enabling examination of the heart. The echocardiography process results in the generation of an echocardiogram (also commonly referred to as an “echo”), which is the image produced.

Echocardiograms may be used to quantify aortic valve stenosis (AS) severity based on, e.g., an aortic valve area (AVA), a mean aortic valve gradient, and a Doppler derived dimensionless index (DI).

A computed tomography (CT) scan is an imaging technique used for imaging internal components of the body. During a CT scan, a series of X-ray images are taken of a section of the body at varying angles, enabling a series of internal cross-sectional images of the body to be generated by a computer.

### SUMMARY

According to an object of the present disclosure, a method of performing hemodynamic assessments of aortic valve stenosis by cardiac computed tomography (CT) scanning is provided. The method may comprise performing a cardiac CT scan of a patient, using a CT scanner. The CT scanner may comprise a gantry comprising one or more X-ray generators, a table configured to support a patient, and a computing device comprising a processor and a memory. The method may comprise, using the computing device, determining, via the CT scan, an aortic valve area (AVA_{CT}) of the patient and a left ventricular outflow tract area (LVOT Area_{CT}) of the patient, calculating an anatomic dimensionless index (DI_{CT}) based on the aortic valve area (AVA_{CT}) and the left ventricular outflow tract area (LVOT Area_{CT}), and determining, based on the aortic valve area (AVA_{CT}), the left ventricular outflow tract area (LVOT Area_{CT}), and the anatomic dimensionless index (DI_{CT}), a severity level of aortic valve stenosis (AS) of the patient.

According to various embodiments, the anatomic dimensionless index, DI_{CT}, may be calculated according to

$${DI}_{CT} = \frac{{AVA}_{CT}}{LVOT {Area}_{CT}} .$$

According to various embodiments, the severity level of the AS of the patient may be determined to be severe when the AVA_{CT} is less than 1.3 cm² and the DI_{CT} is less than 0.25.

According to various embodiments, the severity level of the AS of the patient may be determined to be moderate when the AVA_{CT} is between 1.3 cm² and 2.0 cm² and the DI_{CT} is between 0.25 and 0.45.

According to various embodiments, the method may comprise, using the computing device, computing a mean aortic valve gradient according to:

$$Mean Aortic Value Gradient = 97.364 {x}^{2} - 171.36 x + 77.076 ,$$

wherein x equals an anatomic dimensionless index (DI_{CT}).

According to an object of the present disclosure, a system for performing hemodynamic assessments of aortic valve stenosis by cardiac computed tomography (CT) scanning is provided. The system may comprise a CT scanner configured to perform a cardiac CT scan of a patient. The CT scanner may comprise a gantry comprising one or more X-ray generators and a table configured to support a patient. The system may comprise a computing device comprising a processor and a memory. The computing device may be configured to determine, via the CT scan, an aortic valve area (AVA_{CT}) of the patient and a left ventricular outflow tract area (LVOT Area_{CT}) of the patient, calculate an anatomic dimensionless index (DI_{CT}) based on the aortic valve area (AVA_{CT}) and the left ventricular outflow tract area (LVOT Area_{CT}), and determine, based on the aortic valve area (AVA_{CT}), the left ventricular outflow tract area (LVOT Area_{CT}), and the anatomic dimensionless index (DI_{CT}), a severity level of aortic valve stenosis (AS) of the patient.

According to various embodiments, the computing device may be configured to calculate the anatomic dimensionless index, DI_{CT}, according to

$${DI}_{CT} = \frac{{AVA}_{CT}}{LVOT {Area}_{CT}} .$$

According to various embodiments, the computing device may be configured to determine the severity level of the AS of the patient to be severe when the AVA_{CT} is less than 1.3 cm² and the DI_{CT} is less than 0.25.

According to various embodiments, the computing device may be configured to determine the severity level of the AS of the patient to be moderate when the AVA_{CT} is between 1.3 cm² and 2.0 cm² and the DI_{CT} is between 0.25 and 0.45.

According to various embodiments, the computing device may be configured to compute a mean aortic valve gradient according to:

$$Mean Aortic Value Gradient = 97.364 {x}^{2} - 171.36 x + 77.076 ,$$

wherein x equals an anatomic dimensionless index (DI_{CT}).

According to various embodiments, the gantry may form a bore through which one or more portions of the patient can pass.

According to various embodiments, the table may be configured to move the one or more portions of the patient through the bore.

According to various embodiments, the computing device may be configured to cause the CT scanner to perform the cardiac CT scan of the patient.

According to an object of the present disclosure, a system for performing hemodynamic assessments of aortic valve stenosis by cardiac computed tomography (CT) scanning is provided. The system may comprise a CT scanner configured to perform a cardiac CT scan of a patient. The CT scanner may comprise a gantry comprising one or more X-ray generators and a table configured to support a patient. The system may comprise a computing device comprising a processor and a memory. The memory may be configured to store programming instructions that, when executed by the processor, are configured to cause the processor to determine, via the CT scan, an aortic valve area (AVA_{CT}) of the patient and a left ventricular outflow tract area (LVOT Area_{CT}) of the patient, calculate an anatomic dimensionless index (DI_{CT}) based on the aortic valve area (AVA_{CT}) and the left ventricular outflow tract area (LVOT Area_{CT}), and determine, based on the aortic valve area (AVA_{CT}), the left ventricular outflow tract area (LVOT Area_{CT}), and the anatomic dimensionless index (DI_{CT}), a severity level of aortic valve stenosis (AS) of the patient.

According to various embodiments, the programming instructions, when executed by the processor, may be configured to cause the processor to calculate the anatomic dimensionless index, DI_{CT}, according to

$${DI}_{CT} = \frac{{AVA}_{CT}}{LVOT {Area}_{CT}} .$$

According to various embodiments, the programming instructions, when executed by the processor, may be configured to cause the processor to determine the severity level of the AS of the patient to be severe when the AVA_{CT} is less than 1.3 cm² and the DI_{CT} is less than 0.25.

According to various embodiments, the programming instructions, when executed by the processor, may be configured to cause the processor to determine the severity level of the AS of the patient to be moderate when the AVA_{CT} is between 1.3 cm² and 2.0 cm² and the DI_{CT} is between 0.25 and 0.45.

According to various embodiments, the programming instructions, when executed by the processor, may be configured to cause the processor to compute a mean aortic valve gradient according to:

$$Mean Aortic Value Gradient = 97.364 {x}^{2} - 171.36 x + 77.076 ,$$

wherein x equals an anatomic dimensionless index (DI_{CT}).

According to various embodiments, the gantry may form a bore through which one or more portions of the patient can pass.

According to various embodiments, the programming instructions, when executed by the processor, may be configured to cause the processor to perform the cardiac CT scan of the patient.

According to various embodiments, the table may be configured to move the one or more portions of the patient through the bore.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIG. 1 shows an example computed tomography (CT) scanner, according to various embodiments of the present disclosure;

FIG. 2A shows example CT-generated orthogonal stretched views of an ascending aorta and aortic root and corresponding perpendicular plane demonstrating tips of the aortic valve, according to various embodiments of the present disclosure;

FIG. 2B shows example CT-generated orthogonal stretched views of the ascending aorta and aortic root and corresponding perpendicular plane demonstrating the aortic annulus, according to various embodiments of the present disclosure;

FIG. 2C shows example CT-generated orthogonal stretched views of the ascending aorta and aortic root, and corresponding perpendicular plane demonstrating the aortic annulus, LVOT and aortic valve according to various embodiments of the present disclosure;

FIG. 3 shows a graphical representation of the relationship of the AVA of the cardiac CT scan to the AVA of the echocardiogram, in accordance with an embodiment of the present disclosure;

FIG. 4 shows a graphical representation of the relationship of the DI of the cardiac CT scan to the DI of the echocardiogram, in accordance with an embodiment of the present disclosure;

FIG. 5 shows a graphical representation of the relationship of the DI of the echocardiogram to the mean pressure gradient of the echocardiogram, in accordance with an embodiment of the present disclosure;

FIG. 6 shows a graphical representation of the relationship of the DI of the cardiac CT scan to the mean pressure gradient of the echocardiogram, in accordance with an embodiment of the present disclosure; and

FIG. 7 illustrates example elements of a computing device, according to an exemplary embodiment of the present disclosure.

### DETAILED DESCRIPTION

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

In this document, when terms such as “first” and “second” are used to modify a noun, such use is simply intended to distinguish one item from another, and is not intended to require a sequential order unless specifically stated. In addition, terms of relative position such as “vertical” and “horizontal”, or “front” and “rear”, when used, are intended to be relative to each other and need not be absolute, and only refer to one possible position of the device associated with those terms depending on the device's orientation.

An “electronic device” or a “computing device” refers to a device that includes a processor and memory. Each device may have its own processor and/or memory, or the processor and/or memory may be shared with other devices as in a virtual machine or container arrangement. The memory may contain or receive programming instructions that, when executed by the processor, cause the electronic device to perform one or more operations according to the programming instructions.

The terms “memory,” “memory device,” “computer-readable storage medium,” “data store,” “data storage facility” and the like each refer to a non-transitory device on which computer-readable data, programming instructions or both are stored. Except where specifically stated otherwise, the terms “memory,” “memory device,” “computer-readable storage medium,” “data store,” “data storage facility” and the like are intended to include single device embodiments, embodiments in which multiple memory devices together or collectively store a set of data or instructions, as well as individual sectors within such devices.

The terms “processor” and “processing device” refer to a hardware component of an electronic device that is configured to execute programming instructions. Except where specifically stated otherwise, the singular term “processor” or “processing device” is intended to include both single-processing device embodiments and embodiments in which multiple processing devices together or collectively perform a process.

The term “module” refers to a set of computer-readable programming instructions, as executed by a processor, that cause the processor to perform a specified function.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable programming instructions executed by a processor, controller, or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network-coupled computer systems so that the computer readable media may be stored and executed in a distributed fashion such as, e.g., by a telematics server or a Controller Area Network (CAN).

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

Hereinafter, some embodiments of the present disclosure will be described in detail with reference to the drawings. In the drawings, the same reference numerals will be used throughout to designate the same or equivalent elements. In addition, a detailed description of well-known features or functions will be ruled out in order not to unnecessarily obscure the gist of the present disclosure.

Hereinafter, systems and methods for performing hemodynamic assessments of aortic valve stenosis by cardiac computed tomography, according to embodiments of the present disclosure, will be described with reference to the accompanying drawings.

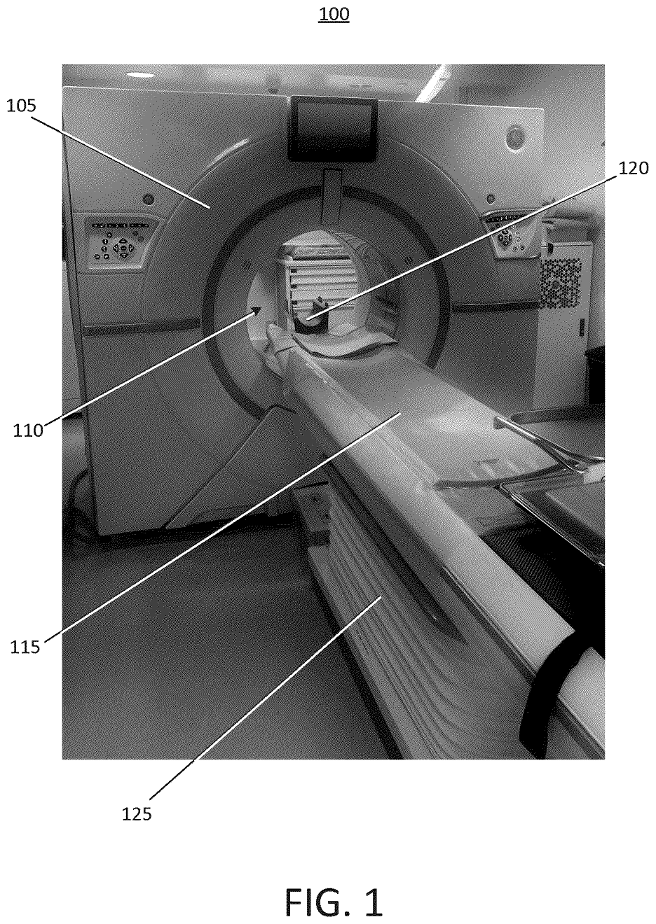

Referring now to FIG. 1, a computed tomography (CT) scanner 100 is illustratively depicted, in accordance with various embodiments of the present disclosure.

According to various embodiments, the CT scanner 100 may comprise a gantry 105, a bore 110, and a table/couch 115.

According to various embodiments, the table/couch 115 may be configured to support a patient during a CT scan procedure. The table/couch 115 may comprise a headrest 120 and/or other components for securing a position of one or more parts of a patient's body. The table/couch 120 may be positioned on a pedestal 125. According to various embodiments, the table/couch 115 may be configured to move, positioning a portion of the patient within the bore 110.

The gantry 105 may comprise one or more X-ray generators configured to generate and direct X-rays towards the patient. According to various embodiments, the gantry 105 may be configured to rotate which the patient is positioned within the bore 110, allowing the X-rays to reach the patient at various angles, enabling the capturing of a CT scan.

A CT scan is an imaging technique, using the CT scanner 100, used for imaging internal components of the body. During a CT scan, a series of X-ray images are taken of a section of the body at varying angles, enabling a series of internal cross-sectional images of the body to be generated by a computer.

CT scans may be performed for imaging various parts of the body. A cardiac CT scan is a CT scan of a patient's heart. Cardiac CT scans are routinely performed to evaluate aortic annular dimension for a transcatheter aortic valve implantation procedure.

While an echocardiogram may be used to quantify a Doppler derived dimensionless index (DI), a cardiac CT scan may be used to derive an anatomic DI based on a continuity equation, according to Equation 1.

$${DI}_{C T} = \frac{{AVA}_{C T}}{LVOT {Area}_{C T}} \qquad Equation 1$$

$${DI}_{ECHO} = \frac{LVOT {VTI}_{ECHO}}{Aortic Valve {VTI}_{ECHO}} \qquad Equation 2$$

Equation 1 is similar to Equation 2, where VTI is a Doppler Velocity Time Integral (VTI) quantified by the echocardiogram, and LVOT is a left ventricular outflow tract.

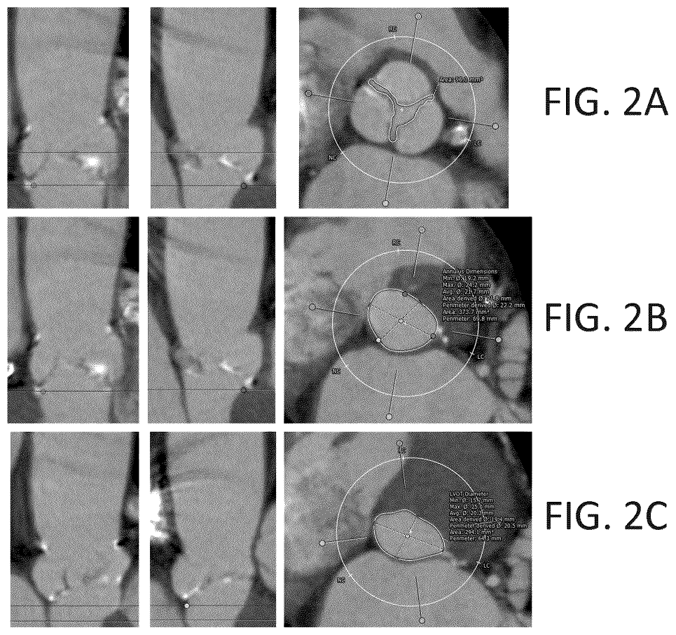

According to various embodiments, a cardiac CT scan may be used to derive an anatomic DI using a cardiac CT-derived aortic annulus measurement, instead of the CT-derived LVOT area, with similar results as shown, e.g., in FIGS. 2A-2C. As shown, e.g., in FIGS. 2A-2C, orthogonal stretched views from a test CT scan are illustratively depicted, in accordance with various embodiments of the present disclosure.

As shown in FIG. 2A, orthogonal stretched views of an ascending aorta and aortic root and corresponding perpendicular plane demonstrating tips of the aortic valve are illustratively depicted. In FIG. 2A, the aortic valve area (AVA) was measured at 0.96 cm², equating to the AVA_{CT}.

As shown in FIG. 2B, orthogonal stretched views of the ascending aorta and aortic root and corresponding perpendicular plane demonstrating the aortic annulus are illustratively depicted. In FIG. 2B, the aortic annulus area, Aortic Annulus_{AREA}, is measured at 3.74 cm².

As shown in FIG. 2C, orthogonal stretched views of the ascending aorta and aortic root, and corresponding perpendicular plane demonstrating the aortic annulus are illustratively depicted. In FIG. 2C, the LVOT area, LVOT_{AREA}, is measured at 2.94 cm².

According to various embodiments, an aortic annulus-derived DI, DI_{Aortic Annulus}, may be obtained using Equation 3.

$${DI}_{Aortic Annulus} = \frac{{AVA}_{C T}}{Aortic Annu {lus}_{AREA}} \qquad Equation 3$$

Using the example measurements obtained in FIGS. 2A-2B, the aortic annulus-derived DI, DI_{Aortic Annulus}, is calculated as

$$0.26 ( \frac{{AVA}_{CT}}{Aortic {Annulus}_{AREA}} = \frac{0.96}{3.74} = 0.26 ) .$$

According to various embodiments, an LVOT-derived DI, DI_{LVOT}, may be obtained using Equation 4.

$${DI}_{L V O T} = \frac{{AVA}_{CT}}{{LVOT}_{AREA}} \qquad Equation 4$$

Using the example measurements obtained in FIGS. 2A and 2C, the LVOT-derived DI, DI_{LVOT}, is calculated as

$$0.33 ( \frac{{AVA}_{CT}}{{LVOT}_{AREA}} = \frac{0.96}{2.94} = 0.33 ) .$$

According to various embodiments, cardiac CT scanning may be used to provide a comprehensive assessment of AS by demonstrating a relationship of DI_{CT} and AVA_{CT} to DI_{ECHO} and an echocardiogram mean pressure gradient.

A retrospective analysis of cardiac CT scans with systolic phase images was performed (where n=86), a maximal AVA and DI were quantified, and echocardiogram results were reviewed. Statistical analysis included descriptive measurements, regression, and correlation analysis.

The retrospective analysis was performed on a cohort of individuals having an average age of 73 years±14 years, with 53% being male. The AVA_{CT} was 2±1.3 cm², the DI_{CT}, using aortic annulus, was 0.40±0.21 with a like LVOT_{CT} derived value of 0.42±0.22.

The echocardiogram demonstrated a left ventricle ejection faction (LVEF) of 57±11%, an AVA of 1.48±1.09 cm², a mean gradient of 29.6±19.8 mmHg, and a DI of 0.41±0.26. The DI_{CT} was similar to DI_{ECHO} (p=NS), but the AVA_{CT} was significantly higher than AVA_{ECHO} (p<0.001).

The DI_{CT} and AVA_{CT} significantly correlated to the DI_{ECHO} and AVA_{ECHO}, with r equal to 0.94 and 0.85, respectively. The DI_{CT} moderately correlated to the mean aortic valve gradient. This suggested that the aortic mean gradient can be estimated from the DI_{CT} using Equation 5.

$$Mean Aortic Value Gradient = 97.364 {x}^{2} - 171.36 x + 77.076 \qquad Equation 5$$

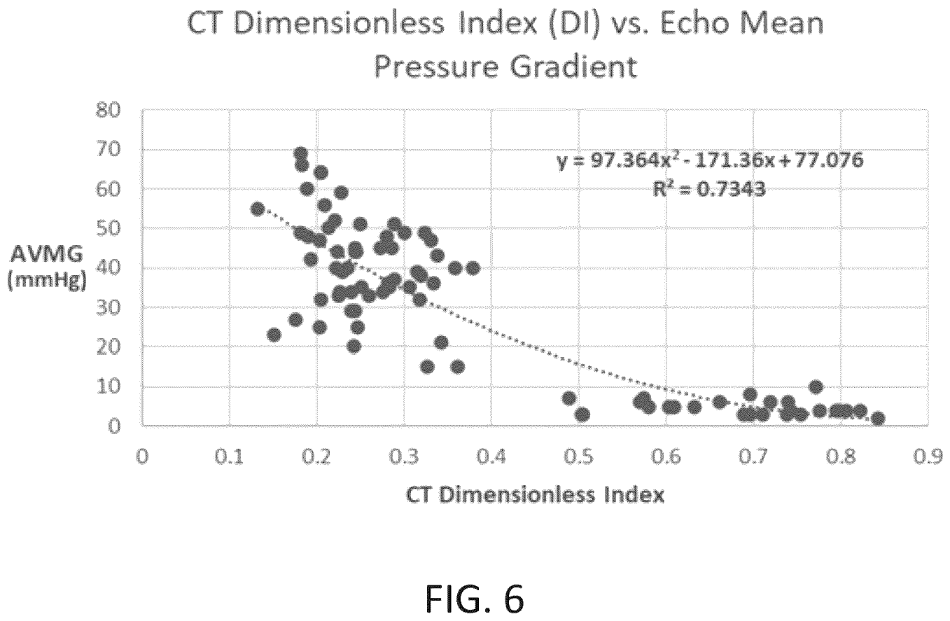

In accordance with FIG. 6, x is the DI_{CT}.

The data indicates that the DI_{CT} and the AVA_{CT} provide a comprehensive assessment of aortic valve stenosis (AS) severity, similar to that of an echocardiogram. AS refers to gradual aortic valve hardening. The heart pumps blood to areas of the body via the aortic valve. This hardening that occurs with AS narrows the aortic valve, decreasing or hindering the heart's ability to pump blood through the aortic valve.

According to various embodiments, AS may be considered severe (a severe severity level) when the AVA_{CT} is less than 1.3 cm² and the DI_{CT} is less than 0.25. These values generally correspond to an AVA_{ECHO} of less than 1.0 cm², a DI_{ECHO} of less than 0.25, and an echocardiogram mean pressure gradient of greater than 40 mm Hg. According to various embodiments, AS may be considered moderate (a moderate severity level) when the AVA_{CT} is generally between 1.3 cm² and 2.0 cm² and the DI_{CT} is generally between 0.25 and 0.45. These values generally correspond to an AVA_{ECHO} of generally between 1 cm² and 1.5 cm², a DI_{ECHO} of generally between 0.25-0.50, and an echocardiogram mean pressure gradient of generally between 20 mm Hg and 40 mm Hg. It is noted, however, that other values for determining AS severity levels may be incorporated, while maintaining the spirit and functionality of the present disclosure.

As shown, e.g., in Table 1, an echocardiogram, a CT (using LVOT), and a CT (using an aortic annulus) may be used to determine a DI, an aortic valve mean pressure gradient (mmHg), and an aortic valve area (AVA) (cm²). Generally, an increased value of the aortic valve mean pressure gradient and/or a decreased value of the AVA is indicative of an increase in AS severity.

TABLE 1

| | | Aortic Valve Mean | |
|---|---|---|---|
| | Dimensionless | Pressure Gradient | Aortic Valve |
| | Index | (mmHg) | Area (cm²) |
| Echocardiogram | 0.16 | 54 | 0.51 |
| CT (using LVOT) | 0.16 | 52 | 0.65 |
| CT (using Aortic | 0.13 | 56 | 0.65 |
| Annulus) | | | |

As shown in Table 1, both the CTs and the echocardiogram are concordant, indicating severe AS in the patient indicated in Table 1.

The echocardiogram-derived aortic mean gradient, the DI_{CT}, and the AVA_{CT} are the current reference standard for the noninvasive evaluation of AS severity. However, echocardiographic evaluation depends on acoustic windows and the ability to obtain Doppler-derived velocity measurements parallel to the flow direction, which can be challenging to obtain. CT is not constrained by these limitations since 3-dimensional (3 D), temporally resolved spatial data is obtained which does not rely on acoustic windows. Therefore, using the CT technique to quantify AS severity of the present disclosure is advantageous over the existing technologies, systems, and techniques in evaluating patients with poor acoustic windows and is an improvement upon existing technologies, systems, and techniques since it is not constrained by the limitations of acoustic windows and the ability to obtain Doppler-derived velocity measurements parallel to the flow direction, solving a long-felt need present in analyzing AS severity in patients with poor acoustic windows.

Additionally, on an echocardiogram, there may be measurement errors of an echocardiogram-derived LVOT area, and errors of velocity gradients across the LVOT and the aortic valve. This may, therefore, result in conflicting values for aortic valve severity based on the estimated mean gradient and the calculated aortic valve area. However, in a CT-derived planimetric aortic valve area, the annulus area and the LVOT area are consistent and accurate, and data indicates that the AVA_{CT} may be correlated to the echocardiogram AVA and that the aortic gradients may be estimated, providing yet another improvement of the present disclosure over the existing technologies, systems, and techniques. This allows for the CT technique of the present disclosure to complement the echocardiographic technique.

There are patients with low aortic gradients despite severe aortic valve stenosis (and are labelled as having low flow low gradient aortic stenosis). Evaluating these patients is challenging. The CT technique of the present disclosure allows for evaluation of these patients and complements the echocardiographic technique. Additionally, the CT technique of the present disclosure may be used in determining AS severity in patients who have undergone the implantation of a prosthetic aortic valve. Evaluation of these patients is typically challenging to complete due to the limited acoustic windows and the reverberation artifact which is generally generated with these patients on the echocardiogram. However, since the CT technique of the present disclosure is not hindered by the limited acoustic windows and the reverberation artifact, the CT technique of the present disclosure again proves advantageous over the existing technologies and techniques.

According to various embodiments, the techniques of the present disclosure may be extended to other cardiac valves such as, but not limited to, the mitral valve, the pulmonic valve, and tricuspid valve to evaluate cardiac valve disorders. Additionally, according to various embodiments, the techniques of the present disclosure may be extended to evaluate patients with hypertrophic cardiomyopathy (patients having thick heart muscle with increased gradient across the LVOT).

According to various embodiments, artificial intelligence (AI) may be incorporated into an approach to estimate aortic pressure and aortic valve area. This will allow for qualitative and quantitative assessment of AS severity similar to the approach outlined above. An additional advantage of this technique may be to embed the AI algorithm in the scanner to triage patients with suspected aortic stenosis.

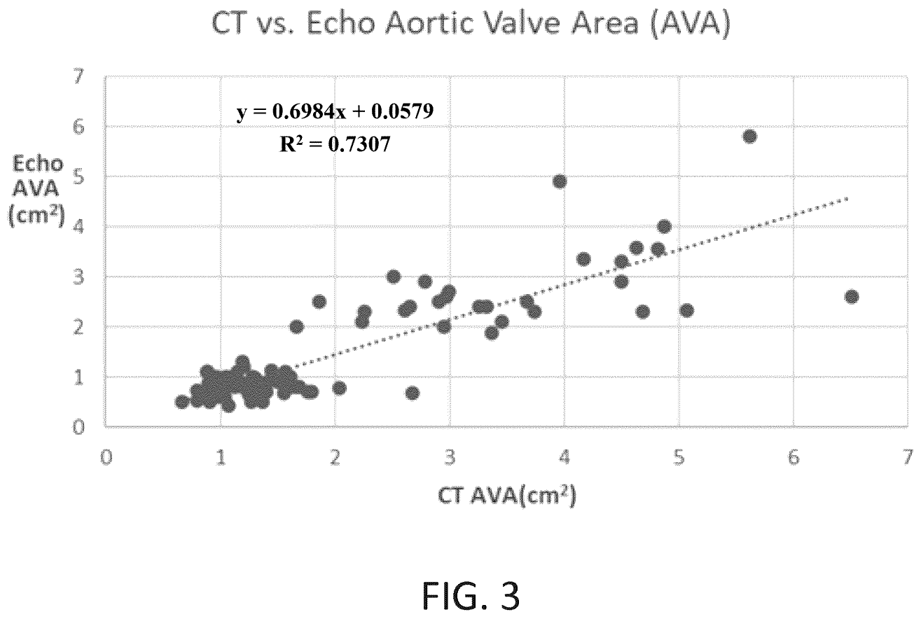

Examples of the various relationships between the echocardiogram and the cardiac CT scan are illustratively depicted in FIGS. 3-6.

As shown in FIG. 3, a graphical representation of the relationship of the AVA of the cardiac CT scan to the AVA of the echocardiogram is illustratively depicted, in accordance with an embodiment of the present disclosure.

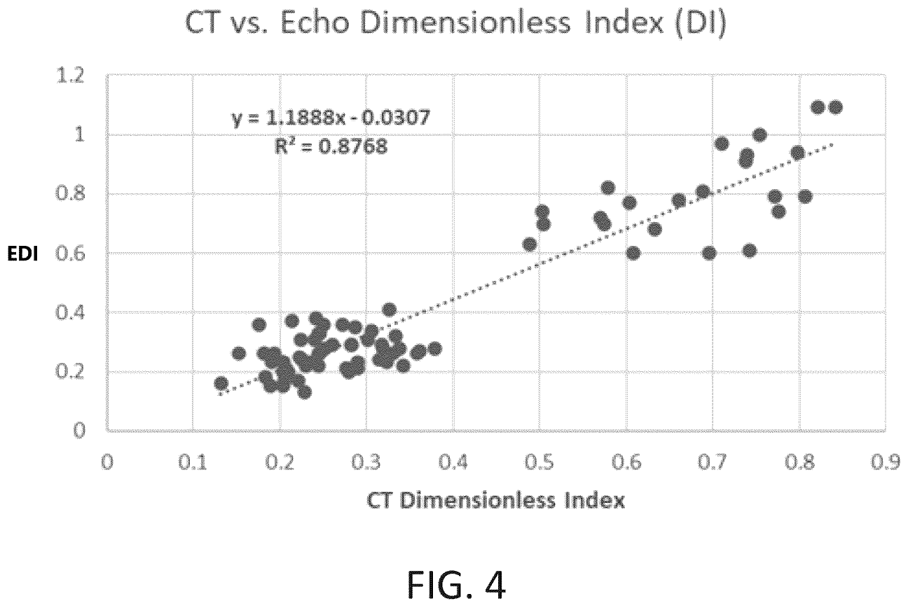

As shown in FIG. 4, a graphical representation of the relationship of the DI of the cardiac CT scan to the DI of the echocardiogram (an Echo Dimensionless Index, or EDI) is illustratively depicted, in accordance with an embodiment of the present disclosure.

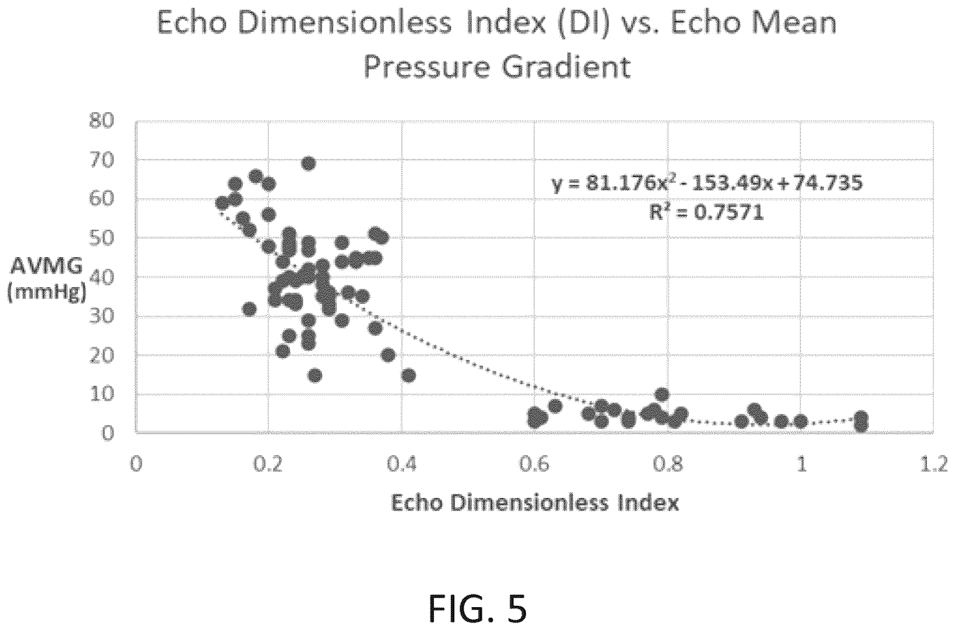

As shown in FIG. 5, a graphical representation of the relationship of the DI of the echocardiogram to the mean pressure gradient of the echocardiogram (the Aortic Valve Mean Gradient, or AVMG) is illustratively depicted, in accordance with an embodiment of the present disclosure.

As shown in FIG. 6, a graphical representation of the relationship of the DI of the cardiac CT scan to the mean pressure gradient of the echocardiogram is illustratively depicted, in accordance with an embodiment of the present disclosure.

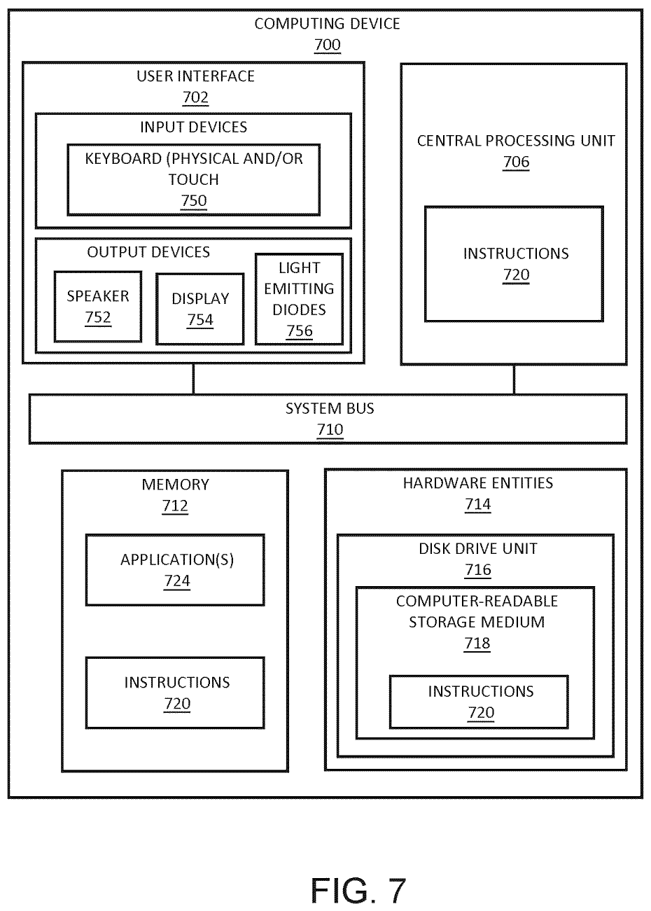

Referring now to FIG. 7, an illustration of an example architecture for a computing device 700 is provided. According to an exemplary embodiment, one or more functions of the present disclosure may be implemented by a computing device such as, e.g., computing device 700 or a computing device similar to computing device 700.

The hardware architecture of FIG. 7 represents one example implementation of a representative computing device configured to perform one or more methods and means for performing hemodynamic assessments of aortic valve stenosis by cardiac computed tomography, as described herein. As such, the computing device 700 of FIG. 7 may be configured to implement at least a portion of the method(s) described herein and/or implement at least a portion of the functions of the system(s) described herein (e.g., CT scanner 100 of FIG. 1). According to various embodiments, the CT scanner 100 of FIG. 1 may comprise one or more computing devices 700. According to various embodiments, the computing device 700 may be configured to cause the CT scanner 100 to perform one or more CT scans (e.g., a cardiac CT scan).

Some or all components of the computing device 700 may be implemented as hardware, software, and/or a combination of hardware and software. The hardware may comprise, but is not limited to, one or more electronic circuits. The electronic circuits may comprise, but are not limited to, passive components (e.g., resistors and capacitors) and/or active components (e.g., amplifiers and/or microprocessors). The passive and/or active components may be adapted to, arranged to, and/or programmed to perform one or more of the methodologies, procedures, or functions described herein.

As shown in FIG. 7, the computing device 700 may comprise a user interface 702, a Central Processing Unit (“CPU”) 706, a system bus 710, a memory 712 connected to and accessible by other portions of computing device 700 through system bus 710, and hardware entities 714 connected to system bus 710. The user interface may comprise input devices and output devices, which may be configured to facilitate user-software interactions for controlling operations of the computing device 700. The input devices may comprise, but are not limited to, a physical and/or touch keyboard 740. The input devices may be connected to the computing device 700 via a wired or wireless connection (e.g., a Bluetooth® connection). The output devices may comprise, but are not limited to, a speaker 742, a display 744, and/or light emitting diodes 746.

At least some of the hardware entities 714 may be configured to perform actions involving access to and use of memory 712, which may be a Random Access Memory (RAM), a disk driver and/or a Compact Disc Read Only Memory (CD-ROM), among other suitable memory types. Hardware entities 714 may comprise a disk drive unit 816 comprising a computer-readable storage medium 718 on which may be stored one or more sets of instructions 720 (e.g., programming instructions such as, but not limited to, software code) configured to implement one or more of the methodologies, procedures, or functions described herein. The instructions 720 may also reside, completely or at least partially, within the memory 712 and/or within the CPU 706 during execution thereof by the computing device 700.

The memory 712 and the CPU 706 may also constitute machine-readable media. The term “machine-readable media”, as used here, refers to a single medium or multiple media (e.g., a centralized or distributed database, and/or associated caches and servers) that store the one or more sets of instructions 720. The term “machine-readable media”, as used here, also refers to any medium that is capable of storing, encoding, or carrying a set of instructions 720 for execution by the computing device 700 and that cause the computing device 700 to perform any one or more of the methodologies of the present disclosure. According to various embodiments, one or more computer applications 724 may be stored on the memory 712.

The features and functions described above, as well as alternatives, may be combined into many other different systems or applications. Various alternatives, modifications, variations or improvements may be made by those skilled in the art, each of which is also intended to be encompassed by the disclosed embodiments.

What is claimed is:

1. A method of performing hemodynamic assessments of aortic valve stenosis by cardiac computed tomography (CT) scanning, comprising:
performing a cardiac CT scan of a patient, using a CT scanner,
wherein the CT scanner comprises:
a gantry comprising one or more X-ray generators;
a table configured to support a patient; and
a computing device comprising a processor and a memory; and
using the computing device:
determining, via the CT scan:
an aortic valve area (AVA_{CT}) of the patient; and
a left ventricular outflow tract area (LVOT Area_{CT}) of the patient;
calculating an anatomic dimensionless index (DI_{CT}) based on the aortic valve area (AVA_{CT}) and the left ventricular outflow tract area (LVOT Area_{CT}); and
determining, based on the aortic valve area (AVA_{CT}), the left ventricular outflow tract area (LVOT Area_{CT}), and the anatomic dimensionless index (DI_{CT}), a severity level of aortic valve stenosis (AS) of the patient.

2. The method of claim 1, wherein the anatomic dimensionless index, DI_{CT}, is calculated according to
$${DI}_{C T} = \frac{{AVA}_{CT}}{LVOT {Area}_{CT}} .$$

3. The method of claim 1, wherein the severity level of the AS of the patient is determined to be severe when the AVA_{CT} is less than 1.3 cm² and the DI_{CT} is less than 0.25.

4. The method of claim 1, wherein the severity level of the AS of the patient is determined to be moderate when the AVA_{CT} is between 1.3 cm² and 2.0 cm² and the DI_{CT} is between 0.25 and 0.45.

5. The method of claim 1, further comprising, using the computing device, computing a mean aortic valve gradient according to:
$$Mean Aortic Value Gradient = 97.364 {x}^{2} - 171.36 x + 77.076 ,$$
wherein x equals an anatomic dimensionless index (DI_{CT}).

6. A system for performing hemodynamic assessments of aortic valve stenosis by cardiac computed tomography (CT) scanning, comprising:
a CT scanner configured to perform a cardiac CT scan of a patient, wherein the CT scanner comprises:
a gantry comprising one or more X-ray generators; and
a table configured to support a patient; and
a computing device comprising a processor and a memory, wherein the computing device is configured to:
determine, via the CT scan:
an aortic valve area (AVA_{CT}) of the patient; and
a left ventricular outflow tract area (LVOT Area_{CT}) of the patient;
calculate an anatomic dimensionless index (DI_{CT}) based on the aortic valve area (AVA_{CT}) and the left ventricular outflow tract area (LVOT Area_{CT}); and
determine, based on the aortic valve area (AVA_{CT}), the left ventricular outflow tract area (LVOT Area_{CT}), and the anatomic dimensionless index (DI_{CT}), a severity level of aortic valve stenosis (AS) of the patient.

7. The system of claim 6, wherein the computing device is configured to calculate the anatomic dimensionless index, DI_{CT}, according to
$${DI}_{C T} = \frac{{AVA}_{CT}}{LVOT {Area}_{CT}} .$$

8. The system of claim 6, wherein the computing device is configured to determine the severity level of the AS of the patient to be severe when the AVA_{CT} is less than 1.3 cm² and the DI_{CT} is less than 0.25.

9. The system of claim 6, wherein the computing device is configured to determine the severity level of the AS of the patient to be moderate when the AVA_{CT} is between 1.3 cm² and 2.0 cm² and the DI_{CT} is between 0.25 and 0.45.

10. The system of claim 6, wherein the computing device is configured to compute a mean aortic valve gradient according to:
Mean Aortic Valve Gradient=97.364x²−171.36x+77.076,
wherein x equals an anatomic dimensionless index (DI_{CT}).

11. The system of claim 6, wherein the gantry forms a bore through which one or more portions of the patient can pass.

12. The system of claim 11, wherein the table is configured to move the one or more portions of the patient through the bore.

13. The system of claim 6, wherein the computing device is configured to cause the CT scanner to perform the cardiac CT scan of the patient.

14. A system for performing hemodynamic assessments of aortic valve stenosis by cardiac computed tomography (CT) scanning, comprising:
a CT scanner configured to perform a cardiac CT scan of a patient, wherein the CT scanner comprises:
a gantry comprising one or more X-ray generators; and
a table configured to support a patient; and
a computing device comprising a processor and a memory, wherein the memory is configured to store programming instructions that, when executed by the processor, are configured to cause the processor to:
determine, via the CT scan:
an aortic valve area (AVA_{CT}) of the patient; and
a left ventricular outflow tract area (LVOT Area_{CT}) of the patient;
calculate an anatomic dimensionless index (DI_{CT}) based on the aortic valve area (AVA_{CT}) and the left ventricular outflow tract area (LVOT Area_{CT}); and
determine, based on the aortic valve area (AVA_{CT}), the left ventricular outflow tract area (LVOT Area_{CT}), and the anatomic dimensionless index (DI_{CT}), a severity level of aortic valve stenosis (AS) of the patient.

15. The system of claim 14, wherein the programming instructions, when executed by the processor, are further configured to cause the processor to calculate the anatomic dimensionless index, DI_{CT}, according to
$${DI}_{C T} = \frac{{AVA}_{CT}}{LVOT {Area}_{CT}} .$$

16. The system of claim 14, wherein the programming instructions, when executed by the processor, are further configured to cause the processor to determine the severity level of the AS of the patient to be severe when the AVA_{CT} is less than 1.3 cm² and the DI_{CT} is less than 0.25.

17. The system of claim 14, wherein the programming instructions, when executed by the processor, are further configured to cause the processor to determine the severity level of the AS of the patient to be moderate when the AVA_{CT} is between 1.3 cm² and 2.0 cm² and the DI_{CT} is between 0.25 and 0.45.

18. The system of claim 14, wherein the programming instructions, when executed by the processor, are further configured to cause the processor to compute a mean aortic valve gradient according to:
$$Mean Aortic Value Gradient = 97.364 {x}^{2} - 171.36 x + 77.076 ,$$
wherein x equals an anatomic dimensionless index (DI_{CT}).

19. The system of claim 14, wherein the gantry forms a bore through which one or more portions of the patient can pass.

20. The system of claim 14, wherein the programming instructions, when executed by the processor, are further configured to cause the processor to perform the cardiac CT scan of the patient.
